(19) United States
(12) Patent Application Publication
(10) Pub. No.: US 2005/0143411 A1
(43) Pub. Date: Jun. 30, 2005

(54) Method for treating pulmonary disease

(75) Inventor: Yoshitaka Hirayama, Osaka (JP)

Correspondence Address: LEYDIG VOIT & MAYER, LTD, 700 THIRTEENTH ST. NW, SUITE 300, WASHINGTON, DC, 20005-3960, US

(73) Assignee: Fujisawa Pharmaceutical Co., Ltd., Osaka-shi (JP)

(21) Appl. No.: 11/012,854

(22) Filed: Dec. 16, 2004

(30) Foreign Application Priority Data
Dec. 30, 2003 (AU) 2003907209
Jan. 20, 2004 (AU) 2004900240

(51) Int. Cl. A61K 31/4745

(52) U.S. Cl. 514/291

(57) ABSTRACT

Methods for treating or preventing airflow obstructions, such as the obstruction induced by cigarette smoke, comprising administering macrolide compounds, such as the FK506 substance and its related compounds, are provided. Compositions containing such compounds are also disclosed.

### CROSS-REFERENCE TO RELATED PATENT APPLICATIONS

This patent application claims the priority under 35 USC 119 of Australian Patent Application Nos. 2003907209, filed Dec. 30, 2003, and 2004900240, filed Jan. 20, 2004, which are each incorporated by reference.

### TECHNICAL FIELD

This invention relates to treating or preventing a pulmonary disease, particularly airflow obstruction, using macrolide compounds.

### BACKGROUND OF THE INVENTION

Airflow obstruction is usually associated with an abnormal inflammatory response of the lungs to noxious particles or gas, which is often accompanying chronic bronchitis and/or emphysema. For example, chronic obstructive pulmonary disease (COPD) is a disease state characterized by airflow obstruction that is not fully reversible.

Currently available information suggests that cigarette smoke-induced lung inflammation has a pathogenic role in the development of COPD.

There are some papers which discuss relationships between COPD and matrix metalloproteinases (MMPs) (e.g., Inflamm. res. 52(2003) 95-100).

WO00/15208 shows a use of some macrolide compounds for treating MMP-mediated disease, particularly cartilage degradation and/or connective tissue degradation such as rheumatoid arthritis.

EP0475994-B1 shows a use of macrolide compounds for treating a reversible obstructive airway disease, such as asthma.

### DETAILED DESCRIPTION OF THE INVENTION

The inventors of this invention have found that the macrolide compounds mentioned below have an activity for treating or preventing airflow obstruction.

Accordingly, this invention provides for treating or preventing airflow obstruction using macrolide compounds.

Additionally, embodiments of the invention provide for treating or preventing one or more of the following: chronic bronchitis, emphysema, and chronic obstructive pulmonary disease.

Further, this invention provides an agent for treating or preventing airflow obstruction, which comprises the macrolide compounds.

Still further, embodiments of the invention provide a method for treating or preventing airflow obstruction, which comprises administering said macrolide compounds to mammals. In an embodiment, the macrolide compound is a tricyclic compound of the following formula (I): 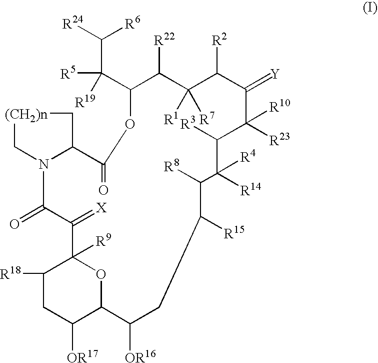
wherein each of adjacent pairs of R¹ and R², R³ and R⁴, and R⁵ and R⁶ independently
(a) is two adjacent hydrogen atoms, but R² may also be an alkyl group or
(b) may form another bond formed between the carbon atoms to which they are attached;
R⁷ is a hydrogen atom, a hydroxy group, a protected hydroxy group, or an alkoxy group, or an oxo group together with R¹;
R⁸ and R⁹ are independently a hydrogen atom or a hydroxy group;
R¹⁰ is a hydrogen atom, an alkyl group, an alkyl group substituted by one or more hydroxy groups, an alkenyl group, an alkenyl group substituted by one or more hydroxy groups, or an alkyl group substituted by an oxo group;
X is an oxo group, (a hydrogen atom and a hydroxy group), (a hydrogen atom and a hydrogen atom), or a group represented by the formula —CH₂O—;
Y is an oxo group, (a hydrogen atom and a hydroxy group), (a hydrogen atom and a hydrogen atom), or a group represented by the formula N—NR¹¹R¹² or N—OR¹³;
R¹¹ and R¹² are independently a hydrogen atom, an alkyl group, an aryl group or a tosyl group;
R¹³, R¹⁴, R¹⁵, R¹⁶, R¹⁷, R¹⁸, R¹⁹, R²² and R²³ are independently a hydrogen atom or an alkyl group;
R²⁴ is an optionally substituted ring system which may contain one or more heteroatoms;
n is an integer of 1 or 2; and
in addition to the above definitions, Y, R¹⁰ and R²³, together with the carbon atoms to which they are attached, may represent a saturated or unsaturated 5- or 6-membered nitrogen, sulfur and/or oxygen containing heterocyclic ring optionally substituted by one or more groups selected from the group consisting of an alkyl, a hydroxy, an alkoxy, a benzyl, a group of the formula —CH₂Se (C₆H₅), and an alkyl substituted by one or more hydroxy groups; or its pharmaceutically acceptable salt.

In a preferred embodiment, the macrolide compound is FK 506 or its hydrate.

In one embodiment, a method for treating airflow obstruction is provided, comprising administering a macrolide compound to a mammal having an airflow obstruction.

In accordance with another embodiment, a method for preventing airflow obstruction comprises administering a macrolide compound to a mammal to prevent an airflow obstruction.

In one embodiment, the airflow obstruction is induced by, or can be induced by, cigarette smoke.

In some embodiments, the macrolide compound is administered to the mammal to treat or prevent one or more of the following conditions: chronic bronchitis, emphysema, and/or chronic obstructive pulmonary disease.

The term “macrolide compounds” for use in accordance with the invention is the generic name of compounds with 12 members or more, which belong to macrocyclic lactones.

As a particular example of the macrolide compounds, the tricyclic compound of the following formula (I) can be exemplified. 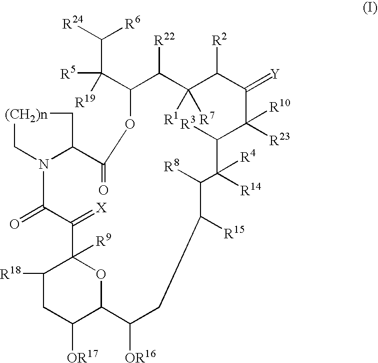
wherein each of adjacent pairs of R¹ and R², R³ and R⁴, and R⁵ and R⁶ independently
(a) is two adjacent hydrogen atoms, but R² may also be an alkyl group or
(b) may form another bond formed between the carbon atoms to which they are attached;
R⁷ is a hydrogen atom, a hydroxy group, a protected hydroxy group, or an alkoxy group, or an oxo group together with R¹;
R⁸and R⁹ are independently a hydrogen atom or a hydroxy group;
R¹⁰ is a hydrogen atom, an alkyl group, an alkyl group substituted by one or more hydroxy groups, an alkenyl group, an alkenyl group substituted by one or more hydroxy groups, or an alkyl group substituted by an oxo group;
X is an oxo group, (a hydrogen atom and a hydroxy group), (a hydrogen atom and a hydrogen atom), or a group represented by the formula —CH₂O—;
Y is an oxo group, (a hydrogen atom and a hydroxy group), (a hydrogen atom and a hydrogen atom), or a group represented by the formula N—NR¹¹R¹² or N—OR¹³;
R¹¹ and R¹² are independently a hydrogen atom, an alkyl group, an aryl group or a tosyl group;
R¹³, R¹⁴, R¹⁵, R¹⁶, R¹⁷, R¹⁸, R¹⁹, R²² and R²³ are independently a hydrogen atom or an alkyl group;
R²⁴ is an optionally substituted ring system which may contain one or more heteroatoms;
n is an integer of 1 or 2; and
in addition to the above definitions, Y, R¹⁰ and R²³, together with the carbon atoms to which they are attached, may represent a saturated or unsaturated 5- or 6-membered nitrogen, sulfur and/or oxygen containing heterocyclic ring optionally substituted by one or more groups selected from the group consisting of an alkyl, a hydroxy, an alkoxy, a benzyl, a group of the formula —CH₂Se (C₆H₅), and an alkyl substituted by one or more hydroxy groups.

The definitions used in the above general formula (I) and the specific and preferred examples thereof are now explained and set forth in detail.

The term “lower” means, unless otherwise indicated, a group having 1 to 6 carbon atoms.

Preferable examples of the “alkyl groups” and an alkyl moiety of the “alkoxy group” include a straight or branched chain aliphatic hydrocarbon residue, for example, a lower alkyl group such as methyl, ethyl, propyl, isopropyl, butyl, isobutyl, pentyl, neopentyl and hexyl.

Preferable examples of the “alkenyl groups” include a straight or branched chain aliphatic hydrocarbon residue having one double-bond, for example, a lower alkenyl group such as vinyl, propenyl (e.g., allyl group), butenyl, methylpropenyl, pentenyl and hexenyl.

Preferable examples of the “aryl groups” include phenyl, tolyl, xylyl, cumenyl, mesityl and naphthyl.

Preferable protective groups in the “protected hydroxy groups” and the “protected amino” are 1-(lower alkylthio)—(lower) alkyl groups such as a lower alkylthiomethyl group (e.g., methylthiomethyl, ethylthiomethyl, propylthiomethyl, isopropylthiomethyl, butylthiomethyl, isobutylthiomethyl, hexylthiomethyl, etc.), more preferably, C₁-C₄ alkylthiomethyl groups, most preferably, methylthiomethyl groups; trisubstituted silyl groups such as a tri (lower) alkylsilyl (e.g., trimethylsilyl, triethylsilyl, tributylsilyl, tert-butyldimethylsilyl, tri-tert-butylsilyl, etc.) or lower alkyl-diarylsilyl (e.g., methyldiphenylsilyl, ethyldiphenylsilyl, propyldiphenylsilyl, tert-butyldiphenyl-silyl, etc.), more preferably, tri (C₁-C₄) alkylsilyl groups and C₁-C₄ alkyldiphenylsilyl groups, most preferably, tert-butyldimethylsilyl groups and tert-butyldiphenylsilyl groups; and an acyl group such as an aliphatic, aromatic acyl group or an aliphatic acyl group substituted by an aromatic group, which are derived from a carboxylic acid, sulfonic acid or carbamic acid.

Examples of the aliphatic acyl groups include a lower alkanoyl group optionally having one or more suitable substituents such as carboxy, e.g., formyl, acetyl, propionyl, butyryl, isobutyryl, valeryl, isovaleryl, pivaloyl, hexanoyl, carboxyacetyl, carboxypropionyl, carboxybutyryl, carboxyhexanoyl, etc.; a cyclo (lower) alkoxy (lower) alkanoyl group optionally having one or more suitable substituents such as lower alkyl, e.g., cyclopropyloxyacetyl, cyclobutyloxypropionyl, cycloheptyloxybutyryl, menthyloxyacetyl, menthyloxypropionyl, menthyloxybutyryl, menthyloxypentanoyl, menthyloxyhexanoyl, etc.; a camphorsulfonyl group; or a lower alkylcarbamoyl group having one or more suitable substituents such as carboxy or protected carboxy, for example, carboxy (lower) alkylcarbamoyl group (e.g., carboxymethylcarbamoyl, carboxyethylcarbamoyl, carboxypropylcarbamoyl, carboxybutylcarbamoyl, carboxypentylcarbamoyl, carboxyhexylcarbamoyl, etc.), tri-(lower)alkylsilyl(lower)alkoxycarbonyl(lower)alkylcarbam oyl group (e.g., trimethylsilylmethoxycarbonylethylcarbamoyl, trimethylsilylethoxycarbonylpropylcarbamoyl, triethylsilylethoxycarbonylpropylcarbamoyl, tert-butyldimethylsilylethoxycarbonylpropylcarbamoyl, tri-methylsilylpropoxycarbonylbutylcarbamoyl, etc.) and soon.

Examples of the aromatic acyl groups include an aroyl group optionally having one or more suitable substituents such as nitro, e.g., benzoyl, toluoyl, xyloyl, naphthoyl, nitrobenzoyl, dinitrobenzoyl, nitronaphthoyl, etc.; and an arenesulfonyl group optionally having one or more suitable substituents such as halogen, e.g., benzenesulfonyl, toluenesulfonyl, xylenesulfonyl, naphthalenesulfonyl, fluorobenzenesulfonyl, chlorobenzenesulfonyl, bromobenzenesulfonyl, iodobenzenesulfonyl, etc.

Examples of the aliphatic acyl groups substituted by an aromatic group include an ar(lower)alkanoyl group optionally having one or more suitable substituents such as lower alkoxy or trihalo (lower) alkyl, e.g., phenylacetyl, phenylpropionyl, phenylbutyryl, 2-trifluoromethyl-2-methoxy-2-phenylacetyl, 2-ethyl-2-trifluoromethyl-2-phenylacetyl, 2-trifluoromethyl-2-propoxy-2-phenylacetyl, etc.

More preferable acyl groups among the aforesaid acyl groups are C₁-C₄ alkanoyl groups optionally having carboxy, cyclo (C₅-C₆) alkoxy (C₁-C₄) alkanoyl groups having two (C₁-C₄) alkyls at the cycloalkyl moiety, camphorsulfonyl groups, carboxy-(C₁-C₄) alkylcarbamoyl groups, tri (C₁-C₄) alkylsilyl (C₁-C₄) alkoxycarbonyl (C₁-C₄)-alkylcarbamoyl groups, benzoyl groups optionally having one or two nitro groups, benzenesulfonyl groups having halogen, or phenyl (C₁-C₄) alkanoyl groups having C₁-C₄ alkoxy and trihalo (C₁-C₄) alkyl group. Among these, the most preferable ones are acetyl, carboxypropionyl, menthyloxyacetyl, camphorsulfonyl, benzoyl, nitrobenzoyl, dinitrobenzoyl, iodobenzenesulfonyl and 2-trifluoromethyl-2-methoxy-2-phenylacetyl.

Preferable examples of the “5- or 6-membered nitrogen, sulfur and/or oxygen containing heterocyclic ring” include a pyrrolyl group and a tetrahydrofuryl group.

R²⁴ is an optionally substituted ring system which may contain one or more heteroatoms. Preferably, R²⁴ may be cyclo (C₅₋₇) alkyl group optionally having suitable substituents, and the following ones can be exemplified.
(a) a 3,4-di-oxo-cyclohexyl group;
(b) a 3-R²⁰-4-R²¹-cyclohexyl group,
   in which R²⁰ is hydroxy, an alkoxy group, an oxo group, or a —OCH₂OCH₂CH₂OCH₃ group, and
      R²¹ is hydroxy, —OCN, an alkoxy group, a heteroaryloxy which may be substituted by suitable substituents, 1- or 2-tetrazolyl, a —OCH₂OCH₂CH₂OCH₃group, a protected hydroxy group, chloro, bromo, iodo, aminooxalyloxy, an azido group, p-tolyloxythiocarbonyloxy, or R²⁵R²⁶CHCOO—,
         in which R²⁵ is optionally protected hydroxy or protected amino, and
            R²⁶ is hydrogen or methyl, or
      R²⁰ and R²¹ together form an oxygen atom in an epoxide ring; or
(c) cyclopentyl group substituted by methoxymethyl, optionally protected hydroxymethyl, acyloxymethyl
   (in which the acyl moiety optionally contains either a dimethylamino group which may be quaternized, or a carboxy group which may be esterified), one or more amino and/or hydroxy groups which may be protected, or aminooxalyloxymethyl. A preferred example is a 2-formyl-cyclopentyl group.

“A heteroaryl which may be substituted by suitable substituents” moiety of the “heteroaryloxy which may be substituted by suitable substituents” may be the ones exemplified for R¹ of the compound of the formula of EP-A-532,088, with preference given to 1-hydroxyethylindol-5-yl, the disclosure of which is incorporated herein by reference.

The tricyclic compounds (I) and their pharmaceutically acceptable salts for use in accordance with this invention are well known to have excellent immunosuppressive activity, antimicrobial activity and other pharmacological activities and, as such, are of value for the treatment or prevention of rejection reactions by transplantation of organs or tissues, graft-vs-host diseases, autoimmune diseases, and infectious diseases (See, EP-A-0184162, EP-A-0323042, EP-A-423714, EP-A-427680, EP-A-465426, EP-A-480623, EP-A-532088, EP-A-532089, EP-A-569337, EP-A-626385, WO89/05303, WO93/05058, WO96/31514, WO91/13889, WO91/19495, WO93/04680, WO93/5059, etc., the disclosures of which are incorporated herein by reference).

Particularly, the compounds which are designated as FR900506 (=FK506), FR900520 (ascomycin), FR900523, and FR900525 are products produced by microorganisms of the genus Streptomyces, such as Streptomyces tsukubaensis No. 9993 [deposited with National Institute of Bioscience and Human Technology Agency of Industrial Science and Technology (formerly Fermentation Research Institute Agency of Industrial Science and Technology), at 1-3, Higashi 1-chome, Tsukuba-shi, Ibaraki, Japan, date of deposit Oct. 5, 1984, accession number FERM BP-927] or Streptomyces hygroscopicus subsp. yakushimaensis No. 7238 [deposited with the National Institute of Bioscience and Human Technology Agency of Industrial Science and Technology (formerly the Fermentation Research Institute Agency of Industrial Science and Technology)], at 1-3, Higashi 1-chome, Tsukuba-shi, Ibaraki, Japan, date of deposit Jan. 12, 1985, accession number FERM BP-928] [EP-A-0184162]. The FK506 (general name: tacrolimus) of the following chemical formula, in particular, is a representative compound. 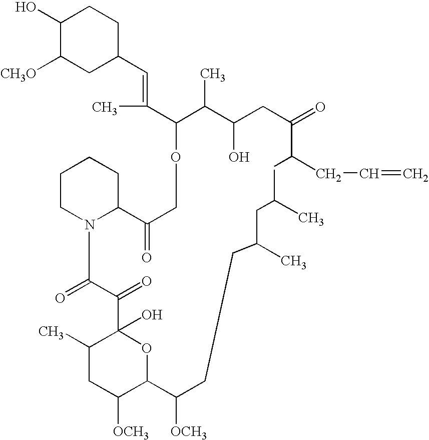
Chemical name:
17-allyl-1,14-dihydroxy-12-[2-(4-hydroxy-3-methoxycyclohexyl)-1-methylvinyl]-23,25-dimethoxy-13,19,21,27-tetramethyl-11,28-dioxa-4-azatricyclo[22.3.1.0^{4,9}]octacos-18-ene-2,3,10,16-tetraone

The preferred examples of the tricyclic compounds (I) are the ones, wherein each of adjacent pairs of R³ and R⁴ or R⁵ and R⁶independently form another bond formed between the carbon atoms to which they are attached;
each of R⁸ and R²³ is independently a hydrogen atom;
R⁹ is a hydroxy group;
R¹⁰ is a methyl group, an ethyl group, a propyl group or an allyl group;
X is (a hydrogen atom and a hydrogen atom) or an oxo group;
Y is an oxo group;
each of R¹⁴, R¹⁵, R¹⁶, R¹⁷, R¹⁸, R¹⁹, and R²² is a methyl group;
R²⁴ is a 3-R²⁰-4-R²¹-cyclohexyl group,
   in which R²⁰ is hydroxy, an alkoxy group, an oxo group, or a —OCH₂OCH₂CH₂OCH₃ group, and
      R²¹ is hydroxy, —OCN, an alkoxy group, a heteroaryloxy which may be substituted by suitable substituents, 1- or 2-tetrazolyl, a —OCH₂OCH₂CH₂OCH₃ group, a protected hydroxy group, chloro, bromo, iodo, aminooxalyloxy, an azido group, p-tolyloxythiocarbonyloxy, or R²⁵R²⁶CHCOO—,
         in which R²⁵ is optionally protected hydroxy or protected amino, and
            R²⁶ is hydrogen or methyl, or R²⁰ and R²¹ together form an oxygen atom in an epoxide ring; and
n is an integer of 1 or 2.

The most preferable tricyclic compounds (I) are, in addition to FK506, ascomycin derivatives such as halogenated-ascomycin (e.g., 33-epi-chloro-33-desoxyascomycin), which is disclosed in EP 427,680, example 66a.

The tricyclic compounds(I) have a similar basic structure, i.e., tricyclic macrolide structure, and at least one or more similar biological properties (for example, immunosupressive activity).

The tricyclic compounds (I) may be in a form of their salts, which include conventional non-toxic and pharmaceutically acceptable salts such as the salt with inorganic or organic bases, specifically, an alkali metal salt such as sodium salt and potassium salt, an alkali earth metal salt such as calcium salt and magnesium salt, an ammonium salt and an amine salt such as triethylamine salt and N-benzyl-N-methylamine salt.

With respect to the macrolide compounds used in the present invention, it is to be understood that there may be conformers and one or more stereoisomers such as optical and geometrical isomers due to asymmetric carbon atom(s) or double bond(s), and such conformers and isomers are also included within the scope of macrolide compound in the present invention. Further, the macrolide compound can be in the form of a solvate, which is included within the scope of the present invention. The solvate preferably includes a hydrate and an ethanolate.

The macrolide compounds usable in the present invention may be administered as pure compounds or mixtures of compounds or preferably, in a pharmaceutical vehicle or carrier.

The pharmaceutical compositions of this invention can be used in the form of a pharmaceutical preparation, for example, in solid, semisolid or liquid form, which contains the macrolide compounds of the present invention, as an active ingredient, in admixture with an organic or inorganic carrier or excipient suitable for external (topical), enteral, intravenous, intramuscular, or parenteral applications. The active ingredient may be compounded, for example, with the usual non-toxic, pharmaceutically acceptable, carriers for tablets, pellets, capsules, eye drops, suppositories, solutions (saline, for example), emulsion, suspensions (olive oil, for example), ointment and any other form suitable for use. The carriers which can be used are water, glucose, lactose, gum acacia, gelatin, mannitol, starch paste, magnesium trisilicate, talc, cornstarch, keratin, colloidal silica, potato starch, urea and other carriers suitable for use in manufacturing preparations, in solid, semisolid, or liquid form, and in addition, auxiliary, stabilizing, thickening and coloring agents and perfumes may be used. The active object compound is included in the pharmaceutical composition in an effective amount sufficient to produce the desired effect upon the process or condition of the disease.

Mammals which may be treated using the method of the present invention include livestock mammals such as cows, horses, etc., domestic animals such as dogs, cats, rats, etc. and humans.

While the dosage of therapeutically effective amount of the macrolide compounds varies from and also depends upon the age and condition of each individual mammal (patient) to be treated, a daily dose of about 0.0001-1000 mg, preferably, 0.001-500 mg, and more preferably, 0.01-100 mg, of the active ingredient is generally given for treating diseases, and an average single dose of about 0.001-0.01 mg, 0.2-0.5 mg, 1 mg, 5 mg, 10 mg, 50 mg, 100 mg, 250 mg and 500 mg is generally administered. Daily doses for chronic administration in humans will be in the range of about 0.1-0.3 mg/kg/day.

Particularly, the tricyclic compound (I) or a pharmaceutically acceptable salt thereof can preferably be administered in an aerosol composition for inhalation, which is, for example, shown by U.S. Pat. No. 6,361,760.

In the form of aerosol composition, the amount of the tricyclic compound (I) or a pharmaceutically acceptable salt is a therapeutically effective one, and varies from and depends on the type of the aerosol composition and the age and condition of each individual patient to be treated. However, it is generally 0.001-10 w/v % and preferably 0.005-5 w/v %.

Other kinds of compounds, such as a 92-agonist, one or more anticholinergic agents, a leukotriene antagonist, corticosteriod, an cromone or an antibiotic, can be administered in admixture of the macrolide compounds of the present invention.

For example, the following compounds are exemplified as preferable ones.

As to a “β2-agonist”, it should be considered to mean any compound which can stimulate the β2 receptor. Preferably, long-acting β2-agonists (such as, salmeterol, formoterol, etc.) and short-acting β2-agonists (such as albuterol, bitolterol, fenoterol, isoetharine, metaproterenol, pirbuterol, terbutaline, salbutamol, etc) can be exemplified. More preferable ones are long-acting β2-agonists, such as, salmeterol, or formoterol.

As to an “anticholinergic agent”, it should be considered to mean any compound which can inhibit cholinergic activity, such as ipratropium bromide, oxitropium bromide, atropine methyl nitrate, atropine sulfate, ipratropium, belladonna extract, scopolamine, scopolamine methobromide, homatropine methobromide, hyoscyamine, isopriopramide, orphenadrine, benzalkonium chloride, tiotropium bromide and glycopyrronium bromide.

As to a “leukotriene antagonist”, Montelukast, [R-(E)]-1-[[[1-[3-[2-(7-Chloro-2- quinolinyl)ethenyl]-phenyl]-3-[2-(1-hydroxy-1-methylethyl)-phenyl]propyl]thio]methyl]cyclopropaneacetic acid (SINGULAIR, Merck & Co., Inc, Rahway, N.J.), which is shown in U.S. Pat. No. 5,565,473, can be exemplified. Other suitable leukotriene antagonists are described in, for example, U.S. Pat. Nos. 4,649,157, 4,845,083, 5,028,615, and 5,244,899.

The following examples illustrate the present invention in further detail, it being to be understood that those examples are not intended to limit the scope of the invention.

### EXAMPLE 1

| | |
|---|---|
| FK 506 Substance | 1 g |
| Hydroxypropyl methylcellulose 2910 (TC-5R) | 1 g |
| Lactose | 2 g |
| Croscarmellose sodium (Ac-Di-Sol) | 1 g |

The FK 506 Substance (1 g) was dissolved in ethanol (10 ml), and thereto was added hydroxypropyl methylcellulose 2910 (TC-5R) (1 g) to prepare a suspension. To this suspension was added dichloromethane (5 ml) to prepare a homogeneous solution. Lactose (2 g) and croscarmellose sodium (Trade Mark: Ac-Di-Sol, maker: Asahi Chemical Industry) were homogeneously suspended to this solution, and then the organic solvent was removed by evaporation. The residual product was dried under reduced pressure for 10 hours by a vacuum dryer, milled for 2 minutes by a coffee mill and then passed through a sieve (32 mesh) to give the solid dispersion composition of FK 506 Substance (5 g). This composition was capsulated by a conventional manner to provide capsules containing 1 mg or 5 mg of FK 506 Substance per each capsule.

### EXAMPLE 2

| | | |
|---|---|---|
| FK506 Substance | 10 | mg |
| HCO-60 | 400 | mg |
| (polyoxyethylenehydrogenated castor oil 60) | | |
| Ethanol | to 1 | ml |

The solution comprising the ingredients stated above is prepared by dissolving the FK506 Substance and HCO-60 in ethanol by a conventional manner. It can be administered via intravenous infusion by diluting with a proper volume of physiological saline.

### EXAMPLE 3

| | |
|---|---|
| FK506 Substance | 10 mg (0.2 (w/v) %) |
| Miglyol 812 | 25 mg (0.5 (w/v) %) |
| HFA-227 | 5 ml |

The FK506 Substance was finely divided to a particle size of 2-3 μm by using a jet mill and the resulting powders were kneaded with Miglyol 812.

After distribution of the kneaded mass, each dispenser was filled with HFA-227 cooled to −20 degree C. beforehand and fitted with a valve to provide an aerosol product containing the following ingredients per unit (5 ml). (cold filling method)

### EXAMPLE 4

| | |
|---|---|
| FK506 Substance | 5 mg |
| Miglyol 812 | 25 mg |
| HFA-134A | 5 ml |

The aerosol composition comprising the above ingredients were prepared in a similar manner to that of Example 4.

### EXAMPLE 5

The effect of the FK506 Substance on the cigarette smoke-induced COPD model in guinea pigs was confirmed in the following manner.

Methods

1. Hartley guinea pigs were exposed to cigarette smoke in a nose-only inhalation chamber for 60 min/day, 5 days/week, for 4 weeks. Animals of the negative control group were exposed to the air.

2. FK506 Substance in the form of a solid dispersion composition, which was prepared in a similar manner to that of Example 1 (above), or its placebo was given orally, after suspension in water, every day about 1 hr before the cigarette smoke exposure.

3. Specific airway resistance (sRaw) was measured as a respiratory function parameter at 0, 1, 2, 3 and 4 weeks after the start of cigarette smoke inhalation by a double body plethysmograph method (Ref. 1).

Reference
Ref. 1; Pennock B E, Cox C P, Rogers R M, Cain W A, Wells J H. A Noninvasive technique for measurement of changes in specific airway resistance. J Appl Physiol. 1979 February; 46(2):399-406.
   Results

The results were summarized in Table 1 shown below.

TABLE 1
Effect of the FK506 Substance on increases in sRaw in
cigarette smoke exposed guinea pigs

| | | sRaw (cmH₂O × mL/(mL/sec)) | | | | | |
|---|---|---|---|---|---|---|---|
| | Dose | | | 1 | 2 | 3 | 4 |
| Group | mg/kg | n | pre | week | weeks | weeks | weeks |
| Air | — | 8 | 1.814 ± 0.112 | 1.944 ± 0.041 | 2.033 ± 0.046 | 2.237 ± 0.043 | 2.335 ± 0.057 |
| Placebo | — | 8 | 1.818 ± 0.056 | 1.912 ± 0.117 | 2.387 ± 0.147# | 2.931 ± 0.143## | 2.883 ± 0.190# |
| FK506 | 1 | 8 | 1.899 ± 0.064 | 1.844 ± 0.123 | 2.012 ± 0.103* | 2.372 ± 0.167* | 2.763 ± 0.276 |
| | 0.32 | 8 | 1.892 ± 0.048 | 1.815 ± 0.073 | 2.089 ± 0.054 | 2.476 ± 0.112 | 2.636 ± 0.133 |

#p < 0.05,
##p < 0.01; Significant difference from the Air group (Student's t-test)
*p < 0.05; Significant difference from the Placebo group (Dunnett's multiple test or Student's t-test)

Inhalation of cigarette smoke caused a significant increase of sRaw in guinea pigs, indicating that cigarette smoke induced an airway obstruction. The orally given FK506 Substance significantly suppressed the decline of the respiratory function.

The above results indicate that the macrolides compounds such as the FK506 Substance are useful for preventing or treating airflow obstruction, more specifically, the airflow obstruction induced by cigarette smoke.

The above results further indicate that the macrolides compounds such as the FK506 Substance are useful for preventing or treating chronic bronchitis and/or emphysema, those of which are characterized by airflow obstruction, and particularly chronic obstructive pulmonary disease characterized by airflow obstruction.

The patents, patent applications and publications cited herein are incorporated by reference.

1. A method for treating airflow obstruction comprising:
administering a macrolide compound to a mammal having an airflow obstruction.

2. A method for preventing airflow obstruction comprising:
administering a macrolide compound to a mammal to prevent an airflow obstruction.

3. The method of claim 1, wherein the airflow obstruction is induced by cigarette smoke.

4. The method of claim 1, wherein the mammal having an airflow obstruction has chronic bronchitis or emphysema.

5. The method of claim 1, wherein the mammal having an airflow obstruction has chronic obstructive pulmonary disease.

6. The method of claim 1, wherein the macrolide compound comprises FK 506 or its hydrate.

7. The method of claim 1, wherein the macrolide compound is a tricyclic compound having the following formula (I): 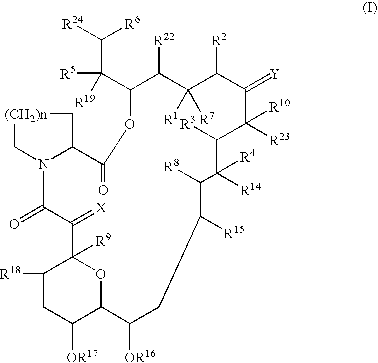
wherein each of adjacent pairs of R¹ and R², R³ and R⁴, and R⁵ and R⁶ independently
(a) is two adjacent hydrogen atoms, but R² may also be an alkyl group or
(b) may form another bond formed between the carbon atoms to which they are attached;
R⁷ is a hydrogen atom, a hydroxy group, a protected hydroxy group, or an alkoxy group, or an oxo group together with R¹;
R⁸and R⁹ are independently a hydrogen atom or a hydroxy group,
R¹⁰ is a hydrogen atom, an alkyl group, an alkyl group substituted by one or more hydroxy groups, an alkenyl group, an alkenyl group substituted by one or more hydroxy groups, or an alkyl group substituted by an oxo group;
X is an oxo group, (a hydrogen atom and a hydroxy group), (a hydrogen atom and a hydrogen atom), or a group represented by the formula —CH₂O—;
Y is an oxo group, (a hydrogen atom and a hydroxy group), (a hydrogen atom and a hydrogen atom), or a group represented by the formula N—NR¹¹R¹² or N—OR¹³;
R¹¹ and R¹² are independently a hydrogen atom, an alkyl group, an aryl group or a tosyl group;
R¹³, R¹⁴, R¹⁵, R¹⁶, R¹⁷, R¹⁸, R¹⁹, R²² and R²³ are independently a hydrogen atom or an alkyl group;
R²⁴ is an optionally substituted ring system which may contain one or more heteroatoms;
n is an integer of 1 or 2; and
in addition to the above definitions, Y, R¹⁰ and R²³, together with the carbon atoms to which they are attached, may represent a saturated or unsaturated 5- or 6-membered nitrogen, sulfur and/or oxygen containing heterocyclic ring optionally substituted by one or more groups selected from the group consisting of an alkyl, a hydroxy, an alkoxy, a benzyl, a group of the formula —CH₂Se (C₆H₅), and an alkyl substituted by one or more hydroxy groups; or its pharmaceutically acceptable salt.

8. The method of claim 2, comprising administering the macrolide compound to prevent chronic bronchitis or emphysema.

9. The method of claim 2, comprising administering the macrolide compound to the mammal to prevent chronic obstructive pulmonary disease.

10. The method of claim 2, comprising administering the macrolide compound to the mammal to prevent airflow obstruction induced by cigarette smoke.
